(19) United States
(12) Patent Application Publication
(10) Pub. No.: US 2008/0077155 A1
(43) Pub. Date: Mar. 27, 2008

(54) SYSTEM AND METHOD FOR DISPLACEMENT OF BONY STRUCTURES

(76) Inventors: Jennifer Diederich, Mansfield, MA (US); Dennis Colleran, North Attleboro, MA (US); Noelle Dye, Charlestown, MA (US)

Correspondence Address: CARR LLP (IST), 670 FOUNDERS SQUARE, 900 JACKSON STREET, DALLAS, TX, 75202, US

(21) Appl. No.: 11/860,485

(22) Filed: Sep. 24, 2007

Related U.S. Application Data
(60) Provisional application No. 60/826,780, filed on Sep. 25, 2006.
(60) Provisional application No. 60/864,357, filed on Nov. 3, 2006.

(51) Int. Cl. A61B 17/58

(52) U.S. Cl. 606/105

(57) ABSTRACT

Disclosed are various embodiments of a displacement device having at least a first arm, a second arm, and a connector. Various embodiments of the connector includes a lateral adjustment mechanism for adjusting the distance between the first arm and the second arm and an angular adjustment mechanism for adjusting the angle between the first arm and the second arm.

### CROSS-REFERENCE TO RELATED APPLICATIONS

This application claims priority to provisional application Ser. No. 60/826,780, “System and Method for Displacement of Bony Structures,” filed Sep. 25, 2006, and provisional application Ser. No. 60/864,357, “System and Method for Displacement of Bony Structures,” filed Nov. 3, 2006, both of which the entire contents are incorporated herein by reference.

This application relates to prior commonly assigned application Ser. No. 10/837,724, “System and Method for Displacement of Bony Structures,” filed May 3, 2004, currently pending, the entire contents of which are incorporated herein by reference. This application relates to prior commonly assigned application Ser. No. 10/690,211, “System and Method for Stabilizing Internal Structures,” filed Oct. 21, 2003, currently pending, the entire contents of which are incorporated herein by reference.

### FIELD OF THE INVENTION

This disclosure relates to devices, instruments, apparatuses, and methods for performing subcutaneous and percutaneous surgery, more particularly, to devices, instruments, apparatuses, and methods for performing minimally invasive spinal surgery.

### BACKGROUND

Patients suffering from orthopedic injuries, deformities, or degenerative diseases often need surgery to stabilize an internal structure, promote healing, or relieve pain. Surgeries to correct spinal problems often involve placing implants such as braces, rods, and various implants between one or more of the patient's vertebrae, anchored into the vertebrae pedicles by screws or hooks. Traditional surgical procedures to correct injuries, defects, and/or abnormalities of the spine have heretofore been substantially invasive. In addition to trauma to the nerves and tissue surrounding the incision, traditional invasive procedures pose significant risk of damage to vital intervening tissues and major muscles and ligaments of the back. The resulting trauma to the tissue and nerves generally requires long recovery periods for the patient and a significant amount of pain experienced during such recovery.

Recently, minimally invasive procedures and micro-surgical procedures have been developed for correction of spinal injuries, defects, and/or abnormalities. These procedures generally involve cutting a small channel down to the affected spinal area and inserting micro-surgical instruments including rod reduction devices into the channel or by using cannulas and the like for receiving instruments therein. Implant engaging instruments such as extensions from the implants may be used for adjustment and manipulation of the implants after the implants have been placed into the bony structures. These percutaneous, minimally invasive and micro-surgical procedures generally cause less disruption to surrounding and intervening tissues and muscles and therefore result in a quicker and less painful recovery period.

Many minimally invasive procedures are practiced for inserting spine stabilization systems to correct defects of the spine. Most spine stabilization systems require implanting bone anchors into vertebrae, the anchors thereafter accompanied by various components such as stabilizing medical implants, which may include rods, braces, connectors, and the like. Often, a surgeon may need to compress or distract bony structures or implants in order to maneuver within the surgical opening and/or correct displacement of vertebrae. Heretofore, available instruments to perform both compression and distraction of bony structures during minimally invasive procedures have been cumbersome or unable to do so without removing the instrument and changing settings, or changing a component, and the like.

### SUMMARY

Certain aspects of the present invention provide methods and apparatuses used in percutaneous and subcutaneous surgical techniques for correcting spinal defects and injuries. There is disclosed certain embodiments of an apparatus for displacing bony structures comprising displacement members which fit over implant engaging instruments such as extensions used to place implants into the bony structures. In certain embodiments, the displacement members may be movably coupled together and may comprise an angular adjustment mechanism and a lateral adjustment mechanism such that the displacement arms may be adjusted both laterally and angularly with respect to each other without the removal or addition of additional parts or instruments. The adjustment of the displacement arms results in adjustment of the extensions and the implants to which they are attached, thereby displacing the bony structures receiving the implants.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of one embodiment of a displacement apparatus incorporating various aspects of the present invention;

FIG. 2A is an exploded perspective view of the displacement apparatus shown in FIG. 1;

FIG. 2B is a perspective view of the lower surface of a connector of the displacement apparatus and components coupled therewith;

FIG. 3A illustrates angular movement of the displacement arms of the displacement apparatus of FIG. 1 with respect to each other;

FIG. 3B illustrates lateral movement of the displacement arms of the displacement apparatus of FIG. 1 with respect to each other;

FIG. 4 is a perspective view of the displacement apparatus of FIG. 1 shown attached over implant engaging instruments in adjacent vertebrae;

FIG. 5 is a perspective view of another embodiment of a displacement apparatus incorporating various aspects of the present invention; and

FIG. 6 is a flowchart illustrating the steps of a method of displacing bony structures.

### DETAILED DESCRIPTION

In the following discussion, numerous specific details are set forth to provide a thorough understanding of the present invention. However, those skilled in the art will appreciate that the present invention may be practiced without such specific details.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

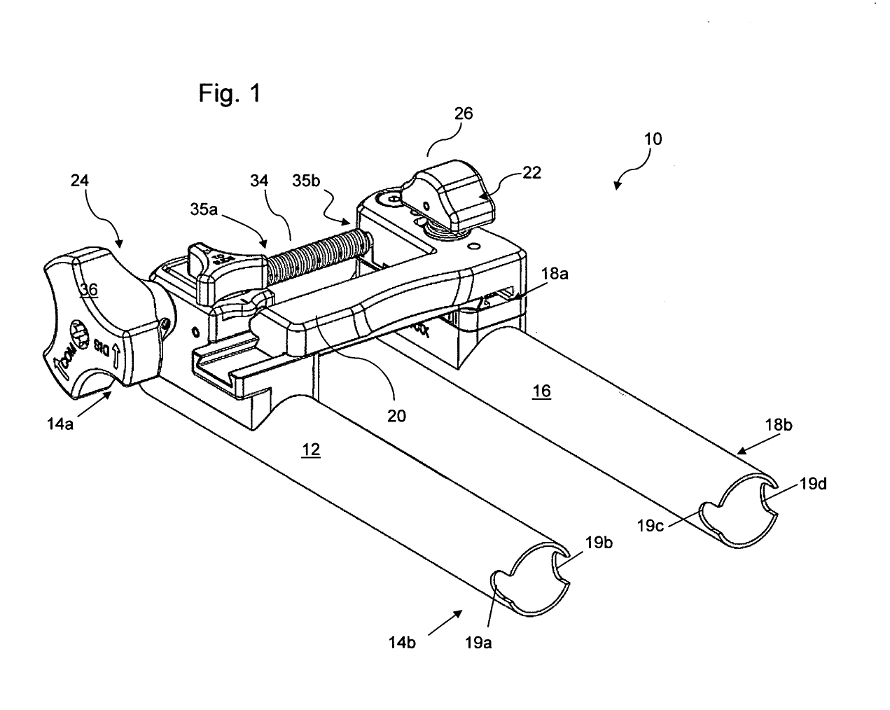

Referring to FIG. 1 there is shown one embodiment of a displacement apparatus 10 which may be used during subcutaneous and percutaneous spinal surgical procedures. The displacement apparatus 10 in the illustrated embodiment may include displacement members such as a first arm or a first guide tube 12 having a proximal end portion 14a and a distal end portion 14b and a second arm or a second guide tube 16 having a proximal end portion 18a and a distal end portion 18b. The proximal end portions 14a and 18a refer to the end portion closer or nearest to the user of the displacement apparatus 10 when the apparatus 10 is in use and the distal end portions 14b and 18b refer to the end portion farthest from the user when the displacement apparatus 10 is in use. The distal end portions 14b and 18b may be adapted to fit over a pedicle screw head and/or connecting rods (not shown). For instance, in one embodiment, a plurality of circular slots 19a and 19b may positioned within the walls of the guide tubes 12 or 16 and be sized to allow a connecting rod (not shown) to fit within their inner surface.

In certain embodiments, the guide tubes 12 and 16 may be movably coupled at the proximal end portions 14a and 18a by a connector 20 having an angular adjustment mechanism 22 on one end thereof and a lateral adjustment mechanism 24 such that the guide tubes 12 and 16 may be adjusted both laterally and angularly with respect to each other without the removal or addition of additional parts or instruments.

In certain embodiments, the angular adjustment mechanism 22 may comprise an angular adjustment user interface such as a button 26 which enables the user of the apparatus 10 to lock the second guide tube 16 from a free movement position into a locked position once the guide tube 16 has been angularly adjusted such that the guide tubes 12 and 16 are positioned to accommodate the patient's natural angular relationship of the bony structures. Once the second guide tube 16 has been angularly adjusted, the first guide tube 12 may be adjusted laterally with respect to the second guide tube 16 in order to compress or distract the bony structures.

In other embodiments, the lateral adjustment mechanism may also comprise a mechanism to move one of the guide tubes laterally relative to the other. Such a mechanism may include a rack and pinion system (not shown) or even a threaded rod 34. In the illustrative embodiment, the threaded rod 34 may comprise a proximal end portion 35a and a distal end portion 35b extending between the first guide tube 12 and the second guide tube 16, the distal end portion 35b referring to the end portion of the rod 34 which engages the proximal end portion 18a of the second guide tube 16. A lateral adjustment user interface such as a knob 36 may be coupled onto the proximal end portion 35a of the threaded rod 34 and used to rotate the threaded rod 34 in order to move the first guide tube 12 laterally with respect to the second guide tube 16.

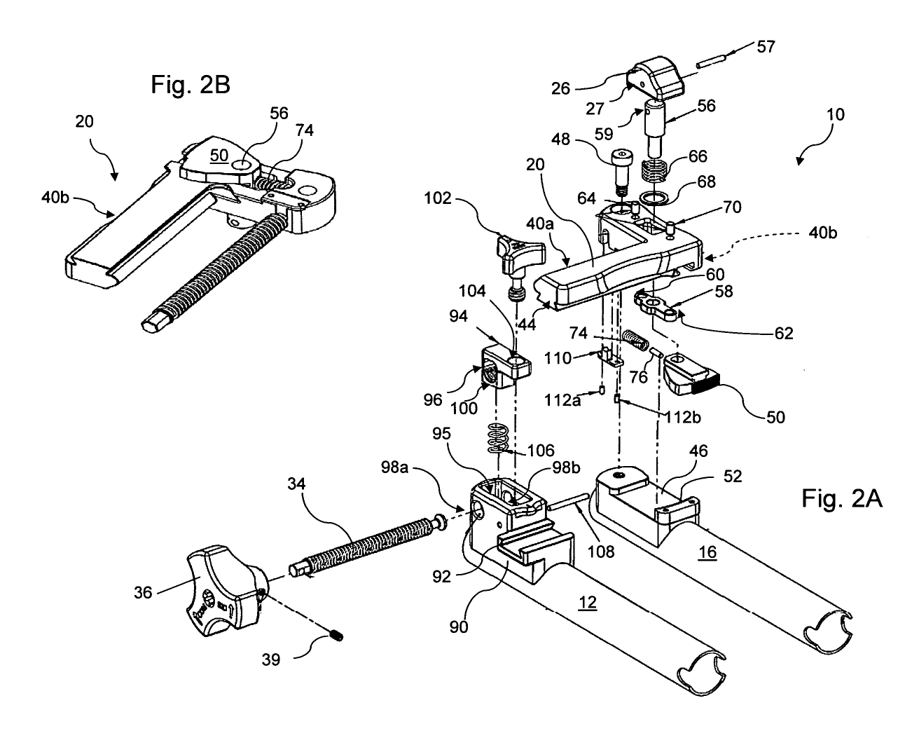

Referring now to FIG. 2A there is an exploded isometric view of one embodiment of the displacement apparatus 10. In this embodiment, the connector 20 may be generally L-shaped and may have an upper surface 40a and a lower surface 40b and a rail 44 positioned on the lower surface 40b. In certain embodiments, the rail 44 extends laterally outward for slidingly coupling with a channel 92 coupled to g the second guide tube 16 with the first guide tube 12. In certain embodiments, the connector 20 may be mounted onto a receiving block 46 on the proximal end portion 18a of the second guide tube 16 and comprise components of the angular adjustment mechanism 22 thereon. In some embodiments, the connector 20 may be secured to the receiving block 46 by a shoulder screw 48 as shown in FIG. 2A, by a bolt, a threaded fastener, or any other suitable fastening means known to those skilled in the art of manufacturing medical instruments.

Secured to the lower surface 40b of the connector 20 may be a pinion 50 of the angular adjustment mechanism 22, which engages a rack 52 mounted onto the receiving block 46. The pinion 50 may be rotatably secured to the lower surface 40b by a locking pin 56 extending through the upper surface 40a of the connector 20. The angular adjustment mechanism further comprises a slide lock 58 secured around the pin 56 just above the pinion 50. The slide lock 58 may comprise a first dowel bore 60 and a second dowel bore 62 enabling the lock 58 to be maintained in either a free motion position or a locked position by the button 26. The button 26 may be coupled with the locking pin 56 by a dowel pin 57 inserted laterally through an orifice 27 through the center of the button 26 and through a corresponding orifice 59 in the top of the locking pin 56. In the free motion position the button 26 rests over and pushes down a first dowel pin 64 into the first dowel bore 60. In the free motion position, the pinion 50 moves freely about the locking pin 56 beneath the lock 58. To lock the pinion 50 in a fixed engagement with the rack 52 the button is depressed and pushed toward the distal end portion of the guide tube 16 engaging and compressing a spring 66 and a locking washer 68. The first dowel pin 64 disengages the first dowel bore 60 and the button 26 is moved over a second dowel pin 70 whereby the second dowel pin 70 engages the second dowel bore 62 to maintain the lock 58 forward. As the slide lock 58 moves forward toward the pinion 50, a compressed spring 74 positioned behind the pinion 50 by a lateral dowel pin 76 is released and maintains the pinion 50 forward into a fixed mating engagement with the rack 52.

Referring now to FIG. 2B there is shown the lower surface 40b of one embodiment of the connector 20. The connector 20 may include a connector bar 21. The connector bar 21 is shown as having an L-shape, however the connector bar 21 may be any suitable shape. In this embodiment, the pinion 50 may be secured about the locking pin 56 and the spring 74 is shown here in the free movement position.

Referring again to FIG. 2A, the lateral adjustment mechanism 24 may comprise a connector block 90 mounted on the first guide tube 12. The connector block 90 may comprise a channel 92 for slidably receiving the rail 44 of the connector 20. The rail 44 and the channel 92 may both be shaped accordingly, such as a dovetail as shown in FIG. 2A, triangular, cylindrical, or any other shape suitable to enable the rail 44 and the channel 92 to slide freely with respect to each other while holding the rail in the channel. The connector block 90 further comprises an opening 95 for receiving a locking block 94 therein. The locking block 94 may comprise a lateral bore 96 which corresponds in size, shape, and location to orifices 98a and 98b in the connector block 90, the bore 96 and orifices for receiving the threaded rod 34 therethrough. The lower inner surface of the bore 96 may comprise threads 100 for engaging the rod 34 during lateral adjustment of the first guide tube 12.

The lateral adjustment mechanism may comprise a free motion position and a lateral adjustment position by use of a user interface such as a threaded knob 102 seated through an orifice 104 in the top of the locking block 94 and received into a threaded bore of the connector block 90. The proximal end portion of the rod 34 is coupled into the interface knob 36 by a dowel pin 39. The distal end portion of the rod rests against a receiving plate 110 coupled into the connector 20 by two dowel pins 112a and 112b. In the free motion position, the threaded knob 102 is fully tightened into the connector block 90 pressing the locking block 94 downward thereby maintaining a spring 106 compressed below the locking block 94. When a user of the apparatus 10 desires to laterally adjust the first guide tube 12, the threaded knob 102 may be turned in a counterclockwise direction until the threads engage a dowel pin 108 in the connector block thereby releasing the locking block 94 upward and uncompressing the spring 106. The uncompressed spring 106 thereby maintains an upward force on the locking block 94 such that the threaded rod 34 engages the threaded bore in the locking block enabling the first guide tube 12 to move laterally with respect to the second guide tube 16 as the lateral interface knob 36 turns and engages the threaded rod 34 to thereby engage the first guide tube 12.

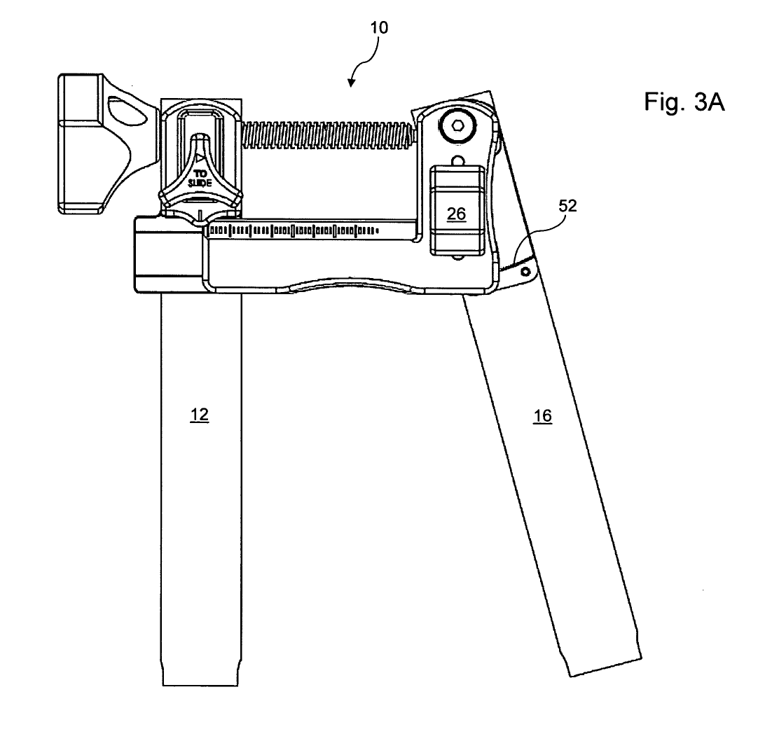

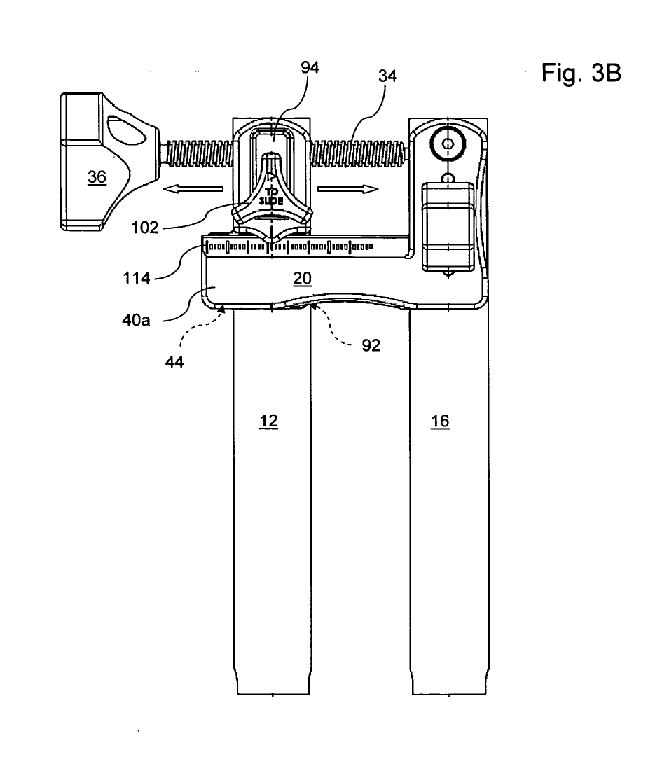

Referring now to FIG. 3A there is shown the displacement apparatus 10 having the second guide tube 16 angularly adjusted with respect to the first guide tube 12. When the apparatus 10 is placed over extensions extending from the implants placed into the bony structures (not shown in FIG. 3A), the angular adjustment mechanism 22 is in the free motion position wherein the pinion 50 rotates freely and is not yet engaged with the rack 52. As the guide tubes are placed over the extensions, the second guide tube 16 will angularly adjust to the placement of the implant engaging extensions which are positioned according to the anatomy of the patient. Once over the extensions and angularly positioned, in certain embodiments, the button 26 may be pressed downward and toward the distal end portion 18b of the second guide tube 16, pushing the pinion 50 forward into engagement with the rack 52. The slide lock 58 is held in a forward position by engagement of the second dowel pin 70 with the second dowel bore 62.

Referring now to FIG. 3B there is shown the displacement apparatus 10 with the first guide tube 12 laterally displaced with respect to the second guide tube 16. When the apparatus 10 is placed over the extensions, the lateral adjustment mechanism 24 is in a free motion position. Once the second guide tube 16 has been locked in an angular position, the first guide tube 12 may be adjusted laterally in order to either compress or distract the bony structures in which the implants have been placed. To begin lateral adjustment, the threaded knob 102 is turned in a counterclockwise direction thereby releasing the compressed spring 106. The spring 106 pushes the locking block 94 upward such that the threads inside the bore 96 engage the threaded rod 34. The user interface knob 36 is then turned either clockwise or counterclockwise to rotate the rod 34 and engage the locking block 94 thereby moving the first guide tube 12 laterally with respect to the second guide tube 16. As the rod 34 engages the locking block 94 the channel 92 slides laterally along the rail 44 of the connector 20 providing a relatively fluid movement of the first guide tube 12. Markings 114 on the upper surface 40a of the connector 20 assist the user of the apparatus with measuring the distance of either compression or distraction.

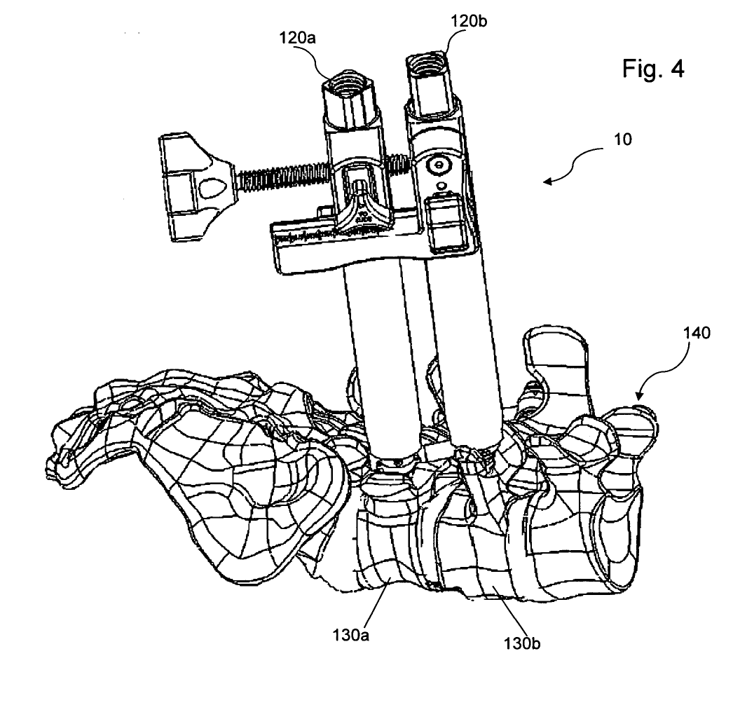

Referring now to FIG. 4, in some embodiments, the guide tubes 12 and 16 of apparatus 10 may be placed over implant engaging instruments such as extensions 120a and 120b which are coupled to pedicle screws or other corrective implants placed into bony structures. As illustrated, the extensions 120a and 120b extend from implants inserted in adjacent vertebrae 130a and 130b of a patient's spine 140. The guide tubes 12 and 16 may thus move the extensions 120a and 120b in order to adjust the placement of the implants in order to compress or distract the bony structures relative to each other as needed.

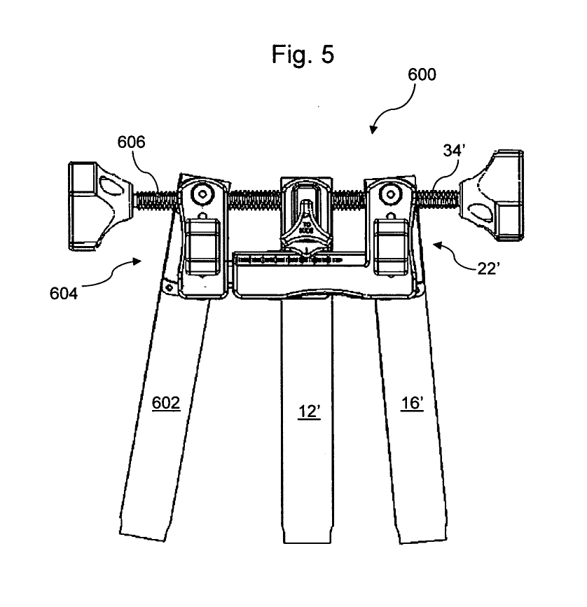

Referring now to FIG. 5 there is displacement apparatus 600 which may comprise an alternate embodiment of the present invention for distracting multi-level implants. Many of the component parts of the displacement apparatus 600 are substantially identical in construction and function to the component parts of the displacement apparatus 10 illustrated in FIGS. 1 through 4 and described hereinabove in conjunction therewith. Such identical components are designated in FIG. 5 with the same reference numerals utilized in the above description of the displacement apparatus 10, but are differentiated therefrom by means of a prime (′) designation.

The displacement apparatus 600 may be used when compression and/or distraction is needed during a procedure to correct the placement of at least three adjacent bony structures. The displacement apparatus 600 differs from the displacement apparatus 10 in that the displacement apparatus 600 may comprise an additional displacement arm such as a third guide tube 602. The third guide tube 602 may be substantially similar to the second guide tube of the displacement apparatus 10 except that the third guide tube 602 may comprise both an angular adjustment mechanism 604 (similar in function and construction to angular adjustment mechanism 22) and some components of a lateral adjustment mechanism thereon, such as a threaded bore for receiving and engaging a threaded rod 606 therein. The displacement apparatus 600 may also differ from the displacement apparatus 10 in that the first guide tube 12′ may be adjustable laterally relative to both a second guide tube 16′ and the third guide tube 602. Both the second guide tube 16′ and the third guide tube 602 may be angularly adjusted relative to the first guide tube 12′ by angular adjustment mechanisms 22′ and 604. Similarly, the second guide tube 16′ may also comprise some components of a lateral adjustment mechanism such as a threaded bore for receiving and engaging a second threaded rod 34′.

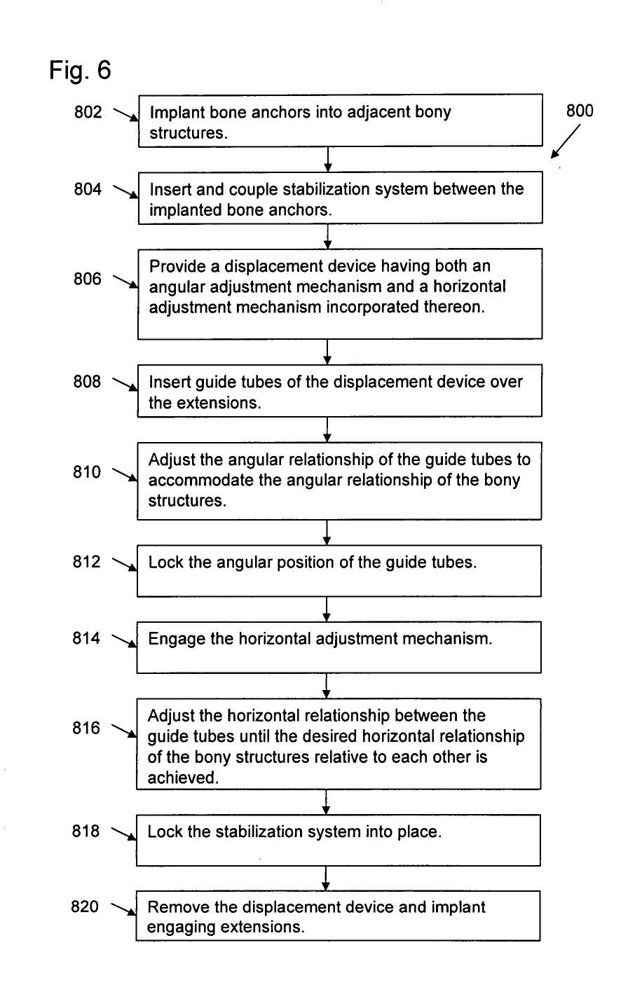

Referring now to FIG. 6, there is shown a flowchart illustrating a method 800 of displacing of bony structures. First, in step 802, bone anchors having implant engaging extensions coupled thereon are implanted into bony structures needing displacement relative to each other, in this example, two adjacent vertebrae. In step 804, a stablizing system such as a rod, or other suitable device or system for stabilization of bony structures, is then implanted between the vertebrae and coupled to the bone anchors. Next, in step 806, a displacement device having both an angular adjustment mechanism and a lateral mechanism incorporated thereon is provided. The displacement device used may be the displacement apparatus 10 described hereinabove. The displacement device may comprise displacement members such as guide tubes which may fit over and engage the implant engaging members extending from the bone anchors. The method 800 may next comprise the step 808 of inserting the guide tubes of the displacement device over the implant engaging extensions. As the guide tubes are placed over the extensions, the guide tubes may be in a free motion position such that a second guide tube moves angularly with respect to a first guide tube. If the displacement device 10 described herein is used, the angular adjustment mechanism may comprise a rack and pinion system wherein the pinion moves freely along the rack coupled onto the second guide tube and is then locked in a desired position by a slide lock. In step 810, the first guide tube may be angularly adjusted with respect to the second guide member as it is placed over the implant engaging mechanisms until a desired angular relationship is achieved to accommodate the angular relationship of the bony structures. The angular position of the guide tubes may then be locked into place in step 812. The method 800 may further comprise the step 814 of engaging a lateral adjustment mechanism and then adjusting the lateral relationship between the guide tubes in step 816 until the desired compression or distraction between the bony structures is achieved. The lateral adjustment mechanism may be engaged by releasing a spring to engage a lateral user interface such as a threaded knob. The threaded knob engages a locking block coupled onto the first guide tube. As the threaded knob is turned, the first guide tube may move laterally with respect to the second guide tube until the desired lateral relationship is achieved. Once the desired lateral relationship between the bony structures is achieved, the method may comprise the step 818 of locking the stabilization system into place to maintain the desired displacement of the bony structures. The method may then comprise the step 820 of removing the displacement device and the implant engaging extensions from the bony structures.

The forgoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

For instance, in some embodiments, there may be an apparatus for displacing bony structures comprising a first and second displacement member, each displacement member having a proximal end portion and a distal end portion, wherein the displacement members are movably coupled together near the proximal end portions thereof; an angular adjustment mechanism for angularly adjusting the displacement arms relative to each other; and a lateral adjustment mechanism for adjusting the displacement arms for laterally adjusting the displacement arms relative to each other. In other embodiments, there may be an apparatus for displacing bony structures comprising a first and second displacement member, each displacement member having a proximal end portion and a distal end portion, wherein the displacement members are movably coupled together near the proximal end portions thereof by a connector; an angular adjustment mechanism for angularly adjusting the displacement arms relative to each other; a lateral adjustment mechanism for adjusting the displacement arms for laterally adjusting the displacement arms relative to each other; wherein the angular adjustment mechanism comprises a rack and pinion and an angular adjustment user interface enabling a user of the apparatus to lock the second displacement member at a desired angular position relative to the first displacement member; wherein the lateral adjustment mechanism comprises an extension for engaging and moving the first displacement member laterally with respect to the second displacement member, the extension having a proximal end portion distal end portion; and a lateral adjustment user interface coupled onto one end of the extension.

In yet other embodiments, there may be an apparatus for the displacement of bony structures wherein the connector is coupled onto the proximal end portion of the second displacement member and comprises a rail which extends laterally outward away from the second displacement member and is slideably received into a channel of the first displacement member.

In still other embodiments, there may be an apparatus for the displacement of bony structures wherein the connector is coupled onto the proximal end portion of the second displacement member and comprises a rail which extends laterally outward away from the second displacement member and is slideably received into a channel of the first displacement member; wherein the connector is coupled onto the second displacement member by a receiving block coupled onto the proximal end portion of the second displacement member.

In other embodiments, there may be an apparatus for the displacement of bony structures comprising a first and second displacement member, each displacement member having a proximal end portion and a distal end portion, wherein the displacement members are movably coupled together near the proximal end portions thereof by a connector; an angular adjustment mechanism for angularly adjusting the displacement arms relative to each other; and a lateral adjustment mechanism for adjusting the displacement arms for laterally adjusting the displacement arms relative to each other.

In another embodiment, there may be a displacement apparatus comprising a first and second displacement member, each displacement member having a proximal end portion and a distal end portion, wherein the displacement members are movably coupled together near the proximal end portions thereof by a connector; an angular adjustment mechanism for angularly adjusting the displacement arms relative to each other; and a lateral adjustment mechanism for adjusting the displacement arms for laterally adjusting the displacement arms relative to each other; wherein the connector is coupled onto the proximal end portion of the second displacement member and comprises a rail which extends laterally outward away from the second displacement member and is slideably received into a channel of the first displacement member.

In yet another embodiment, there may be a displacement apparatus comprising a first and second displacement member, each displacement member having a proximal end portion and a distal end portion, wherein the displacement members are movably coupled together near the proximal end portions thereof by a connector; an angular adjustment mechanism for angularly adjusting the displacement arms relative to each other; and a lateral adjustment mechanism for adjusting the displacement arms for laterally adjusting the displacement arms relative to each other; wherein the connector is coupled onto the proximal end portion of the second displacement member and comprises a rail which extends laterally outward away from the second displacement member and is slideably received into a channel of the first displacement member; wherein the connector is coupled onto the second displacement member by a receiving block coupled onto the proximal end portion of the second displacement member.

In another embodiment, there may be a displacement apparatus wherein wherein the angular adjustment mechanism comprises a receiving block mounted onto the proximal end portion of the second displacement member; a rack mounted onto the receiving block; a pinion rotatably coupled beneath the lower surface of the connector which engages the rack; a slide lock coupled above the pinion wherein the slide lock comprises a first dowel bore and a second dowel bore, the first dowel bore for receiving a first dowel pin for maintaining the slide lock in a first position and a second dowel pin for maintaining the slide lock in a second position; wherein the first position is characterized by the pinion having free motion thereby enabling the second displacement member to have free angular movement with respect to the first displacement member; wherein the second position is characterized by the pinion maintained in a fixed mating engagement with the rack thereby maintaining the second displacement member in a fixed position relative to the first displacement member; and an angular adjustment user interface coupled above the slide lock for moving the slide lock between the first and second positions.

In still another embodiment, there may be a displacement apparatus wherein the lateral adjustment mechanism comprises a connector block mounted onto the proximal end portion of the first guide member; a channel on the connector block for slidably receiving a rail extending from the connector; a locking block mounted atop the connector block; a threaded rod having a proximal and distal end portion extending through a threaded bore of the locking block and engaging the proximal end portion of the second displacement member; a lateral adjustment user interface coupled onto the proximal end portion of the rod; and a locking block engaging mechanism; wherein the locking block engaging mechanism engages the locking block between a first and second position, the first position characterized by the locking block depressed downward such that the rod is not engaging the threads of the threaded bore, the second position characterized by the locking block in a non-depressed condition such that the threaded rod engages the threads of the bore, enabling the locking block, and thus the first displacement member to move laterally with respect to the second displacement member.

There may further be a method of displacing bony structures comprising the steps of implanting bone anchors into adjacent bony structures; implanting and a bone stabilization system and coupling the stabilization system between the bone anchors; coupling implant engaging members to the bone anchors; providing a displacement apparatus having an angular adjustment mechanism and a lateral adjustment mechanism thereon, the displacement device having displacement members for engaging the implant engaging members; inserting the displacement members over the implant engaging members; adjusting the angular relationship of the displacement members until a desired angular position is achieved; locking the displacement members into the desired angular position; engaging the lateral adjustment mechanism of the displacement device; adjusting the lateral relationship of the displacement members until the desired lateral relationship between the bony structures is achieved; locking the stabilization system into place; and removing the displacement device and implant engaging extensions.

What is claimed is:

1. A device for adjusting the distance between bony structures comprising:
a first arm having a longitudinal axis, a proximal end portion and a distal end portion, the distal end portion adapted to couple to a pedicle screw head and having a cutout adapted to accommodate at least a portion of a rod;
a second arm having a longitudinal axis, a proximal end portion and a distal end portion, the distal end portion adapted to couple to a pedicle screw head and having a cutout adapted to accommodate at least a portion of a rod; and
a connector coupling the proximal end portion of the first arm to the proximal end portion of the second arm, the connector having:
an L-shaped connector having a connector bar positioned substantially transverse to the longitudinal axis of the first arm, the connector bar slidingly coupled to the proximal end portion of the first arm and pivotally coupled to the proximal end portion of the second arm;
a lateral adjustment mechanism coupled to the first arm and to the second arm, wherein the lateral adjustment mechanism adjusts the distance between the first arm and second arm; and
an angular adjustment mechanism coupled to the proximal end portion of the second arm and the connector bar for adjusting an angle between the longitudinal axis of the second arm relative to the connector bar.

2. The device of claim 1 further comprising:
a channel coupled to the first arm and having a longitudinal axis substantially transverse to the longitudinal axis of the first arm;
a projection on a surface of the connector bar, wherein the shape of the projection is complementary to the shape of the channel of the first arm;
wherein the channel slidingly receives the projection to provide the sliding coupling between the connector bar and the first arm.

3. The device of claim 1 further comprising:
a biasing mechanism coupled to the lateral advancing mechanism, wherein the biasing mechanism has a first position in which the lateral adjustment mechanism is not capable of moving the first arm relative to the second arm and a second position in which the lateral adjustment mechanism is capable of moving the first arm relative to the second arm.

4. The device of claim 1 wherein the lateral adjustment mechanism comprises a threaded rod system coupled to the first arm and the second arm.

5. The device of claim 1 wherein the lateral adjustment mechanism comprises a rack and pinion system coupled to the first arm and the second arm.

6. The device of claim 1 wherein the angular adjustment mechanism further comprises:
a lock having an unlocked and locked positioned, the lock adapted to prevent the second arm from pivoting relative to the connector bar when in the locked position.

7. A method for adjusting the distance between bony structures comprising:
providing a displacement device having a first arm, a second arm, and an angular adjustment mechanism, a distance advancing mechanism, and a connecting bar connecting the first arm to the second arm in a tongue-in-groove sliding relationship,
unlocking an angular adjustment mechanism of a displacement device;
adjusting angular adjustment mechanism to adjust the angle of the second arm of the displacement device relative to a first arm of the displacement device;
coupling a first arm of the displacement device to a first bone anchor;
coupling a second arm of the displacement device to a second bone anchor;
locking the angular adjustment mechanism after coupling the first and second arms to the first and second anchors;
moving the distance advancing mechanism such that the first arm slides relative to the connecting bar along a longitudinal axis of the connecting bar such that the distance between the first arm and the second arm is adjusted.

8. The method of claim 7 further comprising:
releasing the coupling from the first bone anchor and the second bone anchor after adjusting the distance between the first arm and the second arm of the displacement device.

9. The method of claim 7 further comprising:
coupling a third arm of the displacement device to a third bone anchor.

10. The method of claim 9 further comprising:
adjusting the angle of the third arm of the displacement device relative to the first arm; and
adjusting the lateral distance between the first arm and the third arm.

11. The method of claim 7 wherein adjusting the angle of the second arm occurs before the first arm or the second arm are coupled to the first and second bone anchors.

12. A surgical instrument for adjusting distances between bony structures, the surgical instrument comprising:
a first guide tube;
a second guide tube; and
a connector having:
an L-shaped connector bar coupling the first guide tube to the second guide tube wherein the L-shaped connector bar is pivotally coupled to the second guide tube and slidingly coupled to the first guide tube;
a lateral adjustment means for adjusting a lateral distance between the first and second guide tubes;
an angular adjustment means for adjusting an angular position between the second guide tube and the connector bar; and
an angular fixation means for angularly locking the second guide tube relative to the connector bar.

13. The surgical instrument of claim 12 further comprising:
a locking means having a first position and a second position, wherein in the first position the first guide tube is free to move relative to the second guide tube and in the second position the lateral adjustment means must adjust the lateral distance between the first guide tube and the second guide tube.

14. The surgical instrument of claim 12 wherein the angular adjustment means comprises:
a rack and pinion mechanism.

15. The surgical instrument of claim 12 wherein the lateral adjustment means comprises a threaded rod system coupled to the first arm to the second arm.

16. The surgical instrument of claim 12 wherein the lateral adjustment means comprises a rack and pinion system coupled to the first arm and second arm.

17. The surgical instrument of claim 12 further comprising:
a third guide tube coupled to the L-shaped connector bar;
a second lateral adjustment means for adjusting the distance between the first guide tube and the third guide tube; and
a second angular adjustment means for adjusting the angular position between the third guide tube and the connector bar.

18. A surgical system for adjusting the distance between two bony structures, comprising:
a first and second anchor assembly, each assembly having a bone anchor and a head;
a first extension adapted to coupled to the first pedicle screw assembly;
a second extension adapted to coupled to the second pedicle screw assembly; and
a displacement device having:
a first guide tube adapted to slide over the first extension;
a second guide tube adapted to slide over the second extension; and
a connector having:
an L-shaped connector bar slidingly coupled to the first guide tube and pivotally coupled to the second guide tube;
a lateral adjustment mechanism coupling the first guide tube to the second guide tube, wherein the lateral adjustment mechanism adjusts the distance between the first guide tube and second guide tube; and
an angular adjustment mechanism coupled to the second guide tube and the connector bar, wherein the second guide tube is angularly adjustable relative to the connector bar.

19. The system of claim 18 wherein the system further comprises:
a third anchor assembly having a bone anchor and a head;
a third extension coupled to the third pedicle screw assembly; and
wherein the displacement device further comprises:
a third guide tube adapted to slide over the third extension; wherein the third guide tube is pivotably coupled to the connector bar.

20. The system of claim 19 wherein the displacement device further comprises:
a second lateral adjustment mechanism coupling the third guide tube to the first guide tube, wherein the second distance advancing mechanism adjusts the distance between the first guide tube and the third guide tube.

21. The system of claim 19 wherein the displacement device further comprises:
a second angular adjustment mechanism coupled to the third guide tube and the connector bar, wherein the third guide tube is angularly adjustable relative to the connector bar.

22. The system of claim 18 wherein the displacement device further comprises:
an angle adjustment lock coupled to the second guide tube and adapted to lock the angle of the second guide tube relative to the connector bar.
